(19) United States
(12) Patent Application Publication
(10) Pub. No.: US 2009/0326518 A1
(43) Pub. Date: Dec. 31, 2009

(54) DEVICES AND METHODS FOR MANIPULATING TISSUE

(76) Inventor: Barry H. Rabin, Idaho Falls, ID (US)

Correspondence Address: SPECKMAN LAW GROUP PLLC, 1201 THIRD AVENUE, SUITE 330, SEATTLE, WA, 98101, US

(21) Appl. No.: 12/370,482

(22) Filed: Feb. 12, 2009

Related U.S. Application Data
(60) Provisional application No. 61/028,571, filed on Feb. 14, 2008.
(60) Provisional application No. 61/105,332, filed on Oct. 14, 2008.

(51) Int. Cl. A61B 17/00

(52) U.S. Cl. 606/1

(57) ABSTRACT

The present invention provides new minimally invasive interventional devices and methods for conveniently moving, lifting, positioning, retracting or otherwise manipulating body tissues or organs, while avoiding damage or trauma to these tissues or organs. A manifold is inserted into the patient's body that is deployed and positioned in surface contact with both the target tissue/organ to be manipulated and another moveable structure. The manifold is has at least one evacuation space in communication with at least a portion of the surfaces of each of the target tissue/organ and the moveable structure. A vacuum source external to the patient's body is activated and temporarily and releasably adheres, attaches or otherwise joins the target tissue/organ and moveable structure together. By subsequently manipulating the moveable structure, the target tissue/organ is thereby simultaneously manipulated in the desired manner.

### REFERENCE TO PRIORITY APPLICATIONS

This application claims priority under 35 U.S.C. § 119(e) to U.S. Provisional Patent Application Ser. No. 61/028,571, filed Feb. 14, 2008, and to U.S. Provisional Patent Application Ser. No. 61/105,332, filed Oct. 14, 2008. These patent applications are incorporated herein by reference in their entireties.

### FIELD OF THE INVENTION

The present invention relates generally to interventional devices and methods, and more particularly to minimally invasive interventional devices and methods for moving, lifting, positioning, retracting or otherwise manipulating tissues within a patient.

### BACKGROUND AND DESCRIPTION OF THE PRIOR ART

It is widely recognized that laparoscopic surgery is less invasive than open surgery. In fact, since the late 1980's, laparoscopic surgery has become the standard of care for a variety of common interventional procedures, such as cholecystectomy, diagnostic peritonoscopy, tubal ligation, appendectomy, and hernia repair, among others. Laparoscopy currently involves the injection of low pressure gas (typically CO₂) into the abdominal cavity to effectively inflate said cavity, lifting the abdominal wall away from other internal organs and thereby creating a working space for clinicians to perform the desired diagnostic and therapeutic procedures. Instruments are then inserted through the abdominal wall and into the abdominal cavity at multiple small incision points (typically 3-7) via devices known as trocars that provide instrument access.

Recently, efforts are underway to make laparoscopy even less invasive. Techniques known as single incision laparoscopic surgery (SILS) or single port access (SPA) surgery utilize specially designed trocars through which multiple instruments may be inserted into the patient at a single incision to accomplish the procedure. Endoscopic procedures are also being developed employing devices that enter the body via natural orifices such as the mouth, anus, and vagina, and then pierce through the internal hollow organ walls to access the abdominal cavity to carry out the desired diagnostic and therapeutic procedures. These natural orifice translumenal endoscopic surgery (NOTES) approaches are considered even less invasive, involving no external incisions, less scarring, faster recovery times, etc. The number of minimally invasive laparoscopic, endoscopic and NOTES procedures performed each year is growing rapidly, fueled by the increasing worldwide demand for surgical intervention in general (e.g. for treating epidemics such as obesity and its related co-morbidities) along with patients' desires for better cosmetic results, less pain and scarring, etc.

During surgical intervention, it is often necessary to move, lift, reposition, retract or otherwise manipulate tissues and/or internal organs in order to view and/or treat various areas within the body cavity that would otherwise be difficult to access. Various commercially available devices exist and are well known in the art for manipulating organs. Typically these are simple handheld mechanical instruments such as graspers, retractors, probes, or other blunt instruments capable of moving organs by pushing, pulling, grasping, lifting or otherwise repositioning them. Some organs, such as the liver, stomach, and spleen, can be very challenging to move and lift, as they are voluminous, heavy and difficult to grip, and are easily damaged, or bleed if traumatized. Many of the aforementioned devices (e.g. graspers, blunt probes, and the like) impart high stress concentrations on the tissues/organs and are thus less safe than desired. Other of the aforementioned devices (e.g. retractors) are bulky, cumbersome or otherwise inconvenient for the surgeon to use, and are therefore not suitable for integration into the emerging class of minimally invasive procedures (e.g. SILS, SPA, NOTES procedures) where the tools are smaller, have less structural strength, and they must pass through smaller and sometimes tortuous openings in order to reach the treatment area.

There is therefore a clear need for new interventional methods and devices that are convenient to use, and capable of safely moving, lifting, repositioning or otherwise manipulating heavy, large, or easily damaged body organs. The ability to simply and atraumatically manipulate body organs in minimally invasive laparoscopic, endoscopic and NOTES procedures would be of tremendous benefit to surgeons, patients and health care systems.

### BRIEF SUMMARY OF THE INVENTION

The devices and methods of the present invention represent an entirely new interventional approach for lifting and manipulating the position of body organs to provide unencumbered performance of various diagnostic and therapeutic procedures. The devices and methods of the present invention overcome the above stated shortcomings and limitations of the prior art; specifically these methods and devices are less invasive, more convenient to deploy and use, easier to control, and are significantly safer (i.e. less traumatic to tissues and organs).

The present invention may be best described as a system consisting of a vacuum actuated manifold that is inserted into the body, a vacuum source external to the body, and a vacuum communication member connecting the external vacuum source to said manifold. According to the methods of the present invention, the manifold is first inserted into the body and positioned so at least one portion of a surface of the manifold is in direct contact with at least a portion of the surface of the tissue or organ to be manipulated. At least one other portion of a surface of said manifold is positioned in direct contact with at least one moveable structure which can be easily and controllably manipulated by a clinician, and that is provided and used in conjunction with the device, according to the methods disclosed herein.

The manifold is configured to communicate vacuum (i.e. pressure lower than ambient) to both the tissue/organ to be manipulated and the moveable structure to which it is placed in direct contact. Accordingly, in operation, vacuum is supplied by the external vacuum source and delivered by the vacuum communication member to at least a portion of the manifold, or more typically a space, area or region within the manifold that may be evacuated to a controlled and desired pressure. Communication of the vacuum created within said portion of the manifold to each of the tissue/organ to be manipulated and the moveable structure is typically accomplished by providing one or more ports, openings, holes, passages, and the like, within the surface portions of the manifold in contact with the tissue/organ and the moveable structure. Communication of a controlled vacuum pressure from within the manifold to the tissue/organ and moveable structure generates controllably adjustable holding forces between the surface portions of the manifold in contact with each of the surfaces of the tissue/organ and the moveable structure, respectively, thereby joining or otherwise adhering the manifold to both the organ and the moveable structure surfaces. This effectively attaches the organ and moveable structure together in a temporary and releasable manner (i.e. for as long as the vacuum remains actuated), via the intermediately positioned manifold. Once so joined together, manipulation of the moveable structure by the operator thereby transfers mechanical forces to the target tissue/organ, via the intermediate manifold, allowing the target tissue/organ to be lifted, moved, positioned or otherwise manipulated in the desired manner.

In certain preferred embodiments, the manifold of the present invention is designed to be initially collapsed (e.g. by compressing, folding, rolling, etc.) and is delivered into the body in the collapsed (i.e. pre-deployed) configuration having a reduced profile. Insertion of the collapsed manifold into the body cavity can be accomplished by methods well known in the art, such as via a trocar, tiny laparotomy, or endoscope. Once inside the body cavity, the manifold is expanded (i.e. deployed) and positioned appropriately for subsequent actuation by the user, as described below. Said deployment can be accomplished manually by the operator, or in certain embodiments, the manifold deploys in a self-actuating manner when released from the delivery device, returning to a pre-determined shape as a result of inflation, elastic recovery, the incorporation of mechanical spring elements, shape memory materials, and the like.

In some embodiments the size and geometry of the manifold may be adjusted or selected, either before or during use, according to the size and type of organ/tissue to be manipulated in order to optimize the holding force and ensure safe operation.

The manifold can be rigid, flexible and combinations of the foregoing, and it can be produced from any suitable biocompatible material that may be safely inserted and used within a patient. At least some portions of the manifold, as described below, must be sufficiently air impermeable so as to be capable of withstanding moderate vacuum pressures. Examples of suitable biocompatible materials well known in the art include metals, alloys, thermoplastics, silicones, rubbers, fabrics, and the like, and combinations of the foregoing. The manifold and associated hardware, in whole or in part, may be designed for single patient use, for reposable use, or reusable, and combinations of the foregoing.

In some embodiments, the manifold may be provided as a rigid ring, flexible tube, or expandable balloon, formed into the shape of a loop, doughnut or other similar geometrical shape (though not limited to being circular) so as to provide a central hole or opening that provides the space, area or region within the manifold to be evacuated. In other embodiments, the manifold may be formed in more of a linear, tubular configuration, so as to be configured having one or more central channels or lumens that provide the evacuation space(s). In yet other embodiments, the manifold may be provided in the form of a disk, plate or sheet-like structure produced from a material such as porous matrix, foam, sponge, or the like, and having an air impermeable coating at least partially surrounding the structure so as to be capable of being evacuated internally.

The manifold is generally designed and configured having at least one portion of its surface intended to be positioned in contact with the tissue/organ to be manipulated and at least one portion of its surface designed to be positioned in contact with at least one moveable structure that is provided and used in conjunction with the manifold. Each of said contacting surface portions is further configured having at least one vacuum port, hole, passage or other opening therein that is capable of communicating vacuum between the evacuation space within the manifold and each of the tissue/organ and the moveable structure to which it is placed in contact. Typically, though not necessarily, the contacting surface portions and associated vacuum ports for the tissue/organ to be manipulated and the moveable structure are positioned on opposite-facing sides of the manifold. The size, shape, surface area, etc., of each of the contacting surface portions and associated vacuum ports are optimized to ensure that there is sufficient holding force generated based on the pressure differential established during actuation to securely attach each of said surfaces to the tissue/organ and moveable structure, respectively, while simultaneously distributing these holding forces over a sufficiently large surface area such that stress concentrations that may cause organ trauma or tissue damage are minimized. This method of distributing the mechanical forces needed to hold, lift and manipulate heavy organs or body tissues over a substantially large surface area of contact results in significantly reduced contact stresses compared to prior art devices that necessarily concentrate such stresses, such as graspers, blunt dissectors or metallic mechanical retractor devices. The reduced risk of trauma to tissues and organs during their manipulation is a significant improvement over the prior art.

Accordingly, it is helpful to explain some basic design considerations involved in optimizing the pressure differential and contact surface areas to provide a known, desired lifting force, while distributing these forces over a sufficiently large area such that peak stresses on tissue are kept below a safe threshold. The following simplified calculations provide an example of these design considerations.

Assume the goal is to safely lift a patient's liver that weighs up to 1.0 kg. Further, assume the insufflation pressure (i.e. the positive pressure within the abdominal cavity, relative to atmospheric pressure) is established at 15 mm Hg (2.0 kPa), which is a value typically used in standard laparoscopic procedures. We can calculate the theoretical lifting force as a function of pressure differential (i.e. the difference between the absolute pressure established within the manifold during actuation and the ambient insufflation pressure) for manifolds having different size, i.e. different surface areas of vacuum in contact with the liver. FIG. 1A shows the calculation results, where the curve represents the absolute vacuum pressure needed to lift 1 kg for manifolds having various diameters, assuming that the contact surface area of vacuum in contact with the liver is determined by the diameter of a circular vacuum port that represents the region of contact between the tissue and a ring-shaped manifold such as the manifold described in further detail below (e.g. FIG. 1B). In general, for the devices to be as minimally invasive as possible, it would be preferable to make the device as small as practicable. It would also be desirable to limit the vacuum pressure required to accomplish the mission to only moderate (i.e. relatively low) levels in order to keep the cost and complexity of the vacuum system at a minimum and to minimize the possibility for tissue or organ damage resulting from being in contact with vacuum. For example, it can be seen in FIG. 1A that a circular contact surface area of vacuum having a diameter of 1.5 cm can produce the necessary lifting force at a vacuum pressure of only ˜360 mm Hg, well within the range that is readily achievable by inexpensive and widely available vacuum pumps. Reducing the pressure further below that which is required to lift the organ is not necessary, therefore it may be desirable to include optional components (e.g. valves, switches, sensors and/or other control mechanisms) within the system of the present invention to limit the maximum vacuum pressure to effective and safe levels.

Other design considerations may also need to be factored in. For example, assume that clinical research has indicated it is most desirable not to exceed a maximum direct contact stress on the liver of about 20 kPa (150 mm Hg) in order to avoid undesirable tissue damage and ensure patient safety. This places a constraint on the vacuum pressure that can be safely employed by the system. In this case, it would be advisable not to reduce the absolute vacuum pressure below about 625 mm Hg during manipulation of the liver. According to FIG. 1, it can be seen that the diameter of the contact surface area of vacuum for the manifold should be desirably increased to at least about 2.5 cm to meet both the desired performance and safety requirements.

It should be obvious to those skilled in the art that the above theoretical calculations are highly simplified and it is therefore advisable that further detailed modeling and experimentation be performed to optimize safety and performance for the intended mission before finalizing device design. For example, contact surface areas of vacuum are not likely to remain flat and circular, even when a circular geometry for the vacuum ports is employed because both the manifold and tissues are relatively soft and deformable under pressure. There may also be stretching of tissue, shape variations at the seal edges, variable tissue properties, etc., that need to be taken into account.

As described previously, at least a portion of the space existing between each of said contacting surfaces is configured to be evacuated by being in communication with the external vacuum source. Typically the evacuation space is created by, and its size, shape and other characteristics are determined by design of the manifold, e.g. the manifold size, shape, materials of construction, method of deployment, etc. The portions of the manifold that define the evacuation space and those that form the contacting surfaces may be provided as separate structures comprising a manifold assembly, or the manifold may comprise a single unitary structure that serves both the evacuation space and surface contacting functions. In either case, the manifold is designed and configured to ensure that a user controllable vacuum pressure is achievable and maintainable within the evacuation space. The manifold is further designed and configured to ensure that vacuum created within the evacuation space can be transmitted via one or more vacuum ports positioned on each contacting surface, such that sufficient holding forces are generated to temporarily and releasably attach the manifold to both the tissue/organ and moveable structure. It is often desirable to ensure there is sufficient extra holding force provided while vacuum is maintained to safely and effectively move, lift or otherwise manipulate the tissue/organ and/or moveable structure without unintended leakage of vacuum that may lead to separation or release during such use. Accordingly, the rate of evacuation (i.e. the pumping speed) can also be an important design consideration, requiring optimization of both the pumping capacity of the external vacuum source and the size of the lumen within the vacuum communication member.

In one embodiment, the manifold may be provided in the shape of a ring or other similar geometrical structure having a hole or opening through its central region that forms the evacuation space. In this case, the top- and bottom-most portions of the structure form the contacting surface portions, and the open areas defined within the plane of each contacting surface portion serve as opposite-facing vacuum ports that are essentially contiguous with the central evacuation space. The central hole or opening defining the evacuation space is in vacuum communication with the vacuum communication member, and hence the vacuum source, via at least one vacuum passage incorporated into the manifold structure. Said at least one vacuum passage can be selected from the group consisting of openings, holes, slots, perforations, channels, pores, and combinations of the foregoing. In some cases there may be a single such vacuum passage through the walls of the manifold, while in other cases there may be a plurality of such vacuum passages distributed across a surface. The number, size, shape, orientation and position of the one or more vacuum passages may be optimized in order to control, e.g. the rate of evacuation, the uniformity of holding forces, etc.

In another embodiment, the manifold may be provided in the form of a disk, plate or sheet-like structure, wherein the inside and at least a portion of the upper and lower surfaces are at least partially porous and permeable, capable of transmitting vacuum within its interior, while at least a portion of the external surrounding surfaces are solid or dense, forming an impermeable seal around its perimeter. In this case, the internal portion of the structure itself comprises both the evacuation space and vacuum passages, and the top and bottom portions of the structure form the contacting surfaces having one or more vacuum ports therein.

The vacuum communication member, typically a flexible hose, tube, or the like, is operatively connected between the manifold and external vacuum source (usually positioned outside the body for convenience), passing through the body wall via either the same access site that was used to deliver the manifold into the body cavity or any other convenient access opening.

Once introduced into the body, the manifold is positioned appropriately between the target tissue/organ and the moveable structure. Positioning may be effected using any number of conventional tools, such as a grasper, forceps, probe, or the like. Alternatively, in some embodiments, the devices may optionally incorporate additional components or structures that provide operable mechanisms for assisting with the movement or positioning of the device prior to and during deployment. Examples of such mechanisms include guidewires, articulating joints, remotely steerable motors, permanent magnets, and the like, that may be manipulated either inside or outside the body. In one such embodiment, a permanent magnet incorporated within the manifold may communicate with another permanent magnet located outside the body such that movement of the external magnet by the clinician allows non-contacting movement and positioning of the manifold inside the patient.

The moveable structure used in conjunction with the present invention may be another mechanical system component or instrument provided for such use, and it may be used either internal or external to the patient's body. Alternatively, the moveable structure may actually be part of the patient's body that can be easily moved or otherwise manipulated by the operator during the course of the interventional procedure.

In the case of a mechanical system component, the moveable structure may be introduced into the body cavity along with, and initially attached to the manifold. Alternatively, it may be inserted into the body cavity after the manifold is initially deployed, then brought into contact with and operably attached to the manifold during use. For example, a longitudinal arm, shaft, tube, rod, etc., may be introduced into the body cavity via any convenient access port. The distal end of said device may optionally be configured having a portion that is designed with customized size, shape, surface area, etc. (e.g. a flat surface, curved surface, etc.,) that is intended to readily promote attachment to the manifold when vacuum is actuated.

Alternatively, a device used outside the body, such as a permanent magnet, may be in magnetic field communication with a permanent magnet or other magnetically active component optionally incorporated in, or previously placed in contact with, the manifold. In this manner, movement of the permanent magnet outside the body will produce a non-contacting coupled movement in the manifold, which can aid in positioning the device within the body prior to actuation, and also allow manipulation of the tissue/organ to which the manifold is temporarily attached after vacuum actuation.

In certain other embodiments, the moveable structure of the present invention, to which the manifold is temporarily joined, is another tissue or part of the patient's body that may be manipulated by the surgeon, thereby causing the desired movement of the target tissue/organ to which the manifold is also temporarily joined, as described previously. For example, the manifold may be positioned with one surface in contact with the tissue/organ to be manipulated (e.g. the patient's liver) and another surface in contact with the patient's abdominal wall. Upon actuation of the manifold by supplying vacuum from the vacuum source via the vacuum communication member, the manifold becomes temporarily and releasably joined between the liver and the abdominal wall. Movement of the patient's abdominal wall, e.g. by lifting, will therefore cause the liver to also be simultaneously lifted, providing the surgeon with the desired clinically advantageous positioning and visibility for carrying out the intended diagnostic or therapeutic interventional procedures.

In the case of well established laparoscopic procedures, insufflation is routinely used to inflate the body cavity and lift the abdominal wall, thereby creating operative working space for the surgeon. Therefore, referring to the example above where the surgeon desires to retract the liver by lifting it out of the way, it is possible to first insert the manifold into the patient's body while the body cavity is inflated by insufflation. After deploying the manifold, the operator may then position it by laying it on top of the liver so its bottom surface is in contact with the liver. The surgeon may then decrease the insufflation pressure (i.e. reduce the absolute pressure within the abdominal cavity), partially deflating the body cavity. This lowers the abdominal wall, bringing it into contact with the top surface of the manifold. Upon actuation of the manifold by supplying vacuum thereto, the manifold becomes temporarily and releasably joined to the liver below and the abdominal wall above. Subsequently, the surgeon may again increase the insufflation pressure, re-inflating the body cavity to lift the abdominal wall, simultaneously lifting the manifold and liver joined thereto. In this manner, the liver is safely and completely retracted out of the way, providing the surgeon a clear and unobstructed operative working space.

In the present invention, in many cases only a very small, flexible tube may need to pass through the abdominal wall to serve as the vacuum communication member needed to actuate the device. This tube may be routed in any number of ways that don't necessarily require a dedicated trocar, which advantageously frees up a trocar for use with other instruments. For example, the tube may be routed through a small auxiliary channel that may designed and provided in the trocar. It may also be routed along the outside wall of the trocar, through a separate laparotomy without use of a trocar, etc. Alternatively, in some cases, the vacuum actuated manifold may be sealed off using an optional valve configured as part of the manifold assembly such that after vacuum is actuatingly established inside the patient, the tube may be disconnected and removed while the device remains actuated. Compared to prior art mechanical devices, the present invention may therefore eliminate the need for a separate trocar. It also takes up less space within the operative field and minimizes the possibility of causing inadvertent damage to the liver or surrounding tissues and organs.

Substantially similar methods to those described above for using other tissue or another portion of the patient's body (e.g. the abdominal wall) to serve as the moveable structure of the present invention can also be used when other (non-insufflation) methods for lifting the abdominal wall are employed to create the operative working space.

Beyond using the abdominal wall as the moveable structure, it is also possible to use certain other conveniently manipulated tissues or organs within the body as moveable structures in order to manipulate other tissues/organs that may be in close proximity and temporarily joinable to each other using the vacuum actuated manifold, as described herein.

It should be obvious that a wide variety of target tissues, organs and other body structures may be manipulated using the methods and devices of the present invention. Similarly, an equally wide variety of options exist for providing the necessary moveable structure to be used in conjunction with these devices. Accordingly, there are many potential uses and applications of the present invention in a wide variety of interventional procedures. For example, there are many situations where it may be desirable or simply convenient for a clinician to temporarily and releasably attached one body tissue to either another tissue or an inserted device, in the simplest and safest manner possible. Such other uses and applications are all considered within the scope of the present invention.

While the present invention will be described more fully hereinafter with reference to the accompanying drawings, in which particular embodiments are shown and explained, persons skilled in the art may modify the embodiments herein described while achieving the same methods, functions and results. Accordingly, the descriptions that follow are to be understood as illustrative and exemplary of specific structures, aspects and features within the broad scope of the present invention and not as limiting of such broad scope.

### BRIEF DESCRIPTION OF THE FIGURES

FIG. 1A-1B. (A) Theoretical calculations showing the absolute vacuum pressure required to lift a 1 kg body organ for various diameters of a circular contact surface area; and (B) schematic illustration showing a system for manipulating tissue according to one embodiment of the present invention.

FIG. 2A-2C. Schematic illustrations showing deployment and operation of a device according to one embodiment of the present invention, (A) device inserted and positioned inside the abdominal cavity, (B) abdominal wall lowered and vacuum actuated, and (C) abdominal wall and organ lifted.

FIG. 3A-3B. Schematic illustration showing another embodiment of the present invention, (A) perspective view, and (B) top view.

FIG. 4A-4B. Schematic illustration showing another embodiment of the present invention, (A) deployed top view, and (B) pre-deployed inside delivery catheter.

FIG. 5A-5C. Schematic illustrations showing deployment and operation of a device according to one embodiment of the present invention, (A) close up details of deployed and actuated configuration, (B) inserted and positioned inside the abdominal cavity, and (C) abdominal wall and organ lifted.

FIG. 6A-6B. Schematic illustrations showing optional internal self-expanding structures, (A) example 1, and (B) example 2.

FIG. 7. Schematic illustration showing another embodiment of the present invention.

FIG. 8. Schematic illustration showing use of a system of the present invention in the example of a laparoscopic interventional procedure.

FIG. 9. Schematic illustration showing use of a system of the present invention in the example of a natural orifice translumenal endoscopic surgery (NOTES) interventional procedure.

FIG. 10. Schematic illustration showing another embodiment of the present invention.

FIG. 11. Schematic illustration showing another embodiment of the present invention.

### DETAILED DESCRIPTION OF THE INVENTION

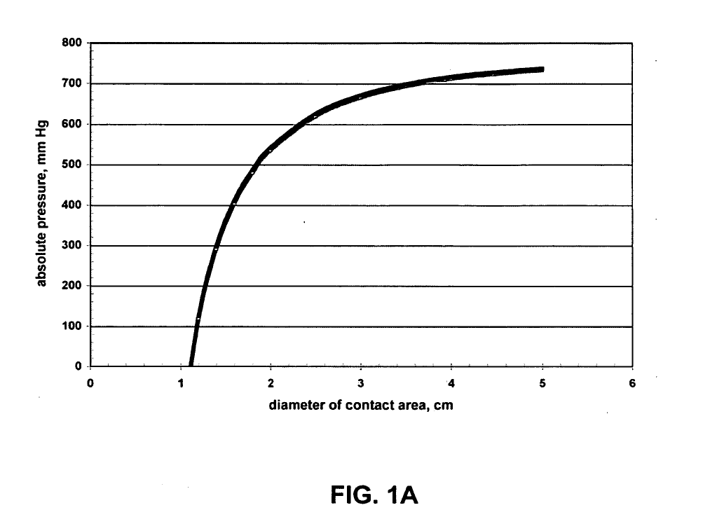

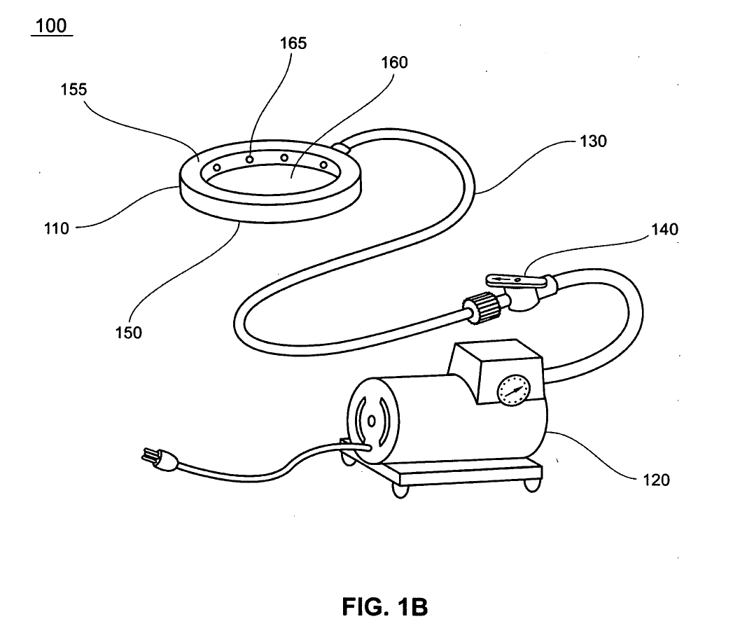

An exemplary system of the present invention is shown schematically in FIG. 1B. System 100 comprises vacuum actuated manifold 110 that is designed to be inserted into a patient's body, vacuum source 120 that typically remains outside the patient's body, and vacuum communication member 130 which is operatively connected between vacuum source 120 and manifold 110. Optionally, the system may contain a variety of connections, fittings, valves, etc., well known to those skilled in the art for interconnecting and assembling vacuum components and systems. For example, as shown in FIG. 1B, system 100 incorporates valve 140 that allows vacuum to manifold 110 to be actuatingly turned on and off by the operator. Ring-shaped manifold 110 is designed and configured having lower surface 150 that is used to establish substantial contact with the organ to be manipulated, and upper surface 155 that used to establish substantial contact with a moveable structure to be provided during use. The central opening in manifold 110 forms an evacuation space 160 that is operatively evacuated by the vacuum source during actuation via a plurality of vacuum passages 165 positioned along the inner surface of evacuation space 160, wherein vacuum passages 165 are interconnected with a hollow channel (not shown) inside manifold 110 that is further operatively interconnected with vacuum communication member 130. In this embodiment, the circular openings at the bottom and top of the manifold, defined by the planar area inside the lines of contact along lower surface 150 and upper surface 155, provide the vacuum ports associated with each of the contacting surfaces that communicate vacuum from evacuation space 160 to each of the target tissue/organ and moveable structure, respectively.

In the example shown, manifold 110 consists of a rigid ring produced from a commercially available biocompatible thermoplastic material that may be manufactured by methods well known in the art, such as injection molding, machining, and the like. Manifold 110 may be produced having a wide variety of sizes and shapes, depending on and optimized for the specific intended use. However, in general, it is desirable to minimize the overall size of the device, consistent with providing sufficient holding forces for the intended use, while minimizing the potential for tissue damage and organ trauma by maximizing the available tissue surface contact areas. Accordingly, the outer diameter of manifold 110 is preferably between 0.1 cm and 30 cm, more preferably between 0.5 cm and 20 cm, and most preferably between 1 cm and 10 cm. Based on the outer diameter, the inner diameter of manifold 110 may be designed accordingly to provide the desired volume of evacuation space 160 and the desired size (i.e. surface area) of the vacuum ports, which controls the vacuum contact area and hence the holding forces produced during actuation (as described in FIG. 1A).

Vacuum communication member 130 is typically provided as a flexible hose or conduit produced from a commercially available biocompatible thermoplastic material capable of vacuum use, along with associated fittings, connections, switches, sensors, control valves, etc. well known to those skilled in the art of vacuum systems. The outer and inner diameters of vacuum communication member 130 may vary considerably depending on the size of manifold 110 and evacuation space 160, as well as the desired rate of evacuation, desired maximum achievable vacuum pressure, desire to overcome small vacuum leakage in actual practice, etc. In general, the outer diameter of vacuum communication member 130 is preferably between 0.01 cm and 2 cm, more preferably between 0.05 cm and 1 cm, and most preferably between 0.1 cm and 0.5 cm.

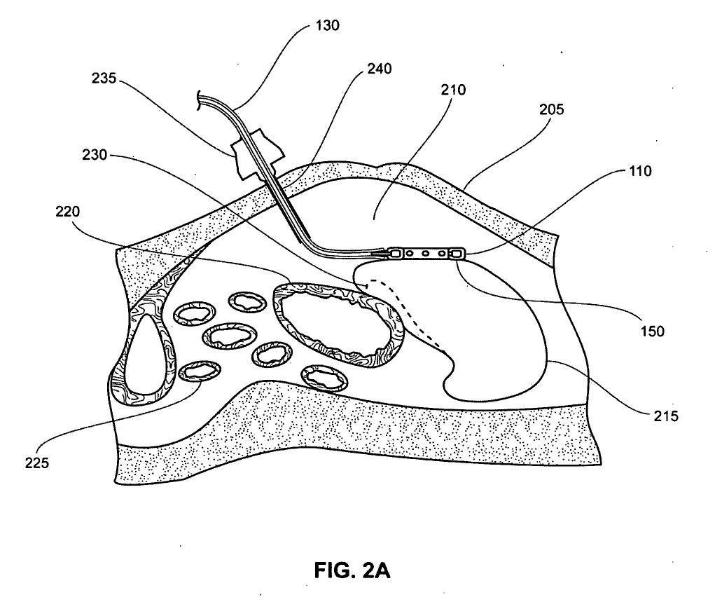

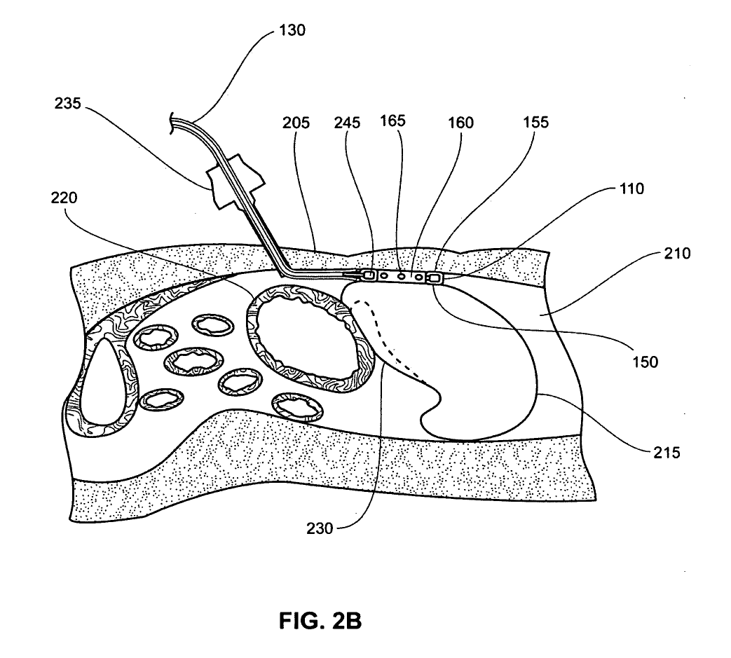

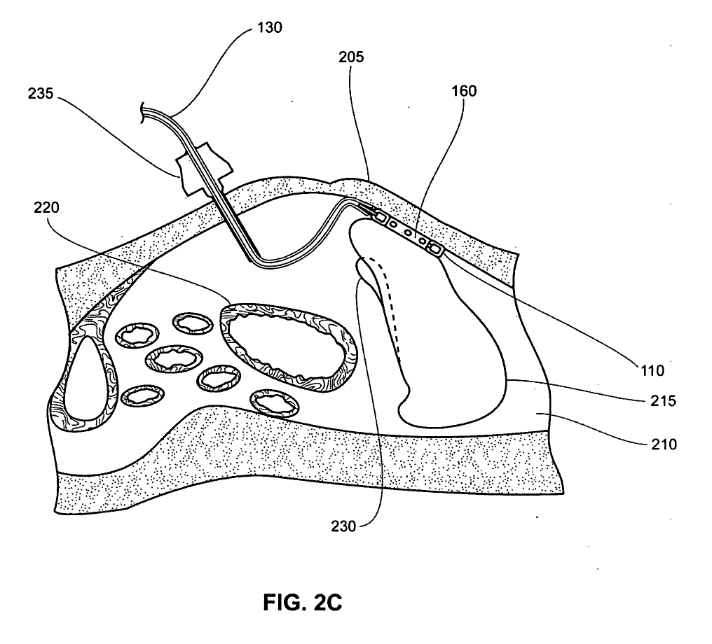

FIG. 2 illustrates operation of exemplary system 100 according to the methods of the present invention. By way of example, it is assumed in FIG. 2 that the surgeon desires to perform a laparoscopic cholecysectomy, a common interventional procedure in which the patient's gall bladder is removed. In FIG. 2A, a cross section of a typical patient's anatomy is schematically shown, wherein abdominal wall 205 has been lifted via CO₂ insufflation, a method commonly used to establish pneumoperitoneum (i.e. a pressurized gas-filled working space) within abdominal cavity 210. The patient's liver 215 is shown positioned in typical relationship to the stomach 220 and gastrointestinal tract 225. As will be appreciated by those skilled in the art, in the normal condition, gall bladder 230 is rather inconveniently positioned between the underside of the liver and the anterior and/or cephalic aspect of the stomach, making it effectively inaccessible without retraction of the liver. Also shown in FIG. 2A is trocar 235 that has been operatively inserted through abdominal wall 205 at incision 240 that was made in order to gain access to abdominal cavity 210. As shown, in the initial deployment of system 100, manifold 110, which is connected to an vacuum source positioned remotely (not shown) by vacuum communication member 130 has been inserted into abdominal cavity 210 and positioned with lower surface 150 in substantial contact with the exposed and readily accessible anterior aspect of liver 215.

As shown in FIG. 2B, in the next step of deployment of system 100, the CO₂ insufflation pressure has been released, allowing abdominal wall 205 to lower toward it's normal position. This brings upper surface 155 of manifold 110 into substantial contact with abdominal wall 205, which serves as the moveable structure in the present example. Actuation of the remote vacuum source (not shown) reduces the pressure inside vacuum communication member 130, which is operatively connected to hollow channel 245 and vacuum passages 165. This reduces the pressure inside evacuation space 160 to a level that is selectively controlled (either manually by the operator or at least partially automatically by control mechanisms that may be optionally incorporated into system 100) to a value that transmits sufficient holding forces to cause temporary attachment of liver 215 to lower surface 150 and abdominal wall 205 to upper surface 155. In this manner, liver 215 becomes effectively and temporarily attached to abdominal wall 205 via manifold 110.

The range of vacuum pressures needed during actuation, which determines the required capabilities and ratings for external vacuum source 120, the materials of construction and dimensions of the various system components, etc., depends on the dimensions of manifold 110, the contacting surface areas of vacuum provided by the vacuum ports on lower surface 150 and upper surface 155, as well as the design requirement for the interventional mission (e.g. the weight of the organ to be lifted, desired safety factor, etc.), as explained previously with reference to FIG. 1A. All of these factors are considered adjustable variables and design parameters that may be optimized within the scope of the present invention. In general, for most organ manipulation applications, the working vacuum pressure (absolute) required during actuation is preferably less than 760 mm Hg, more preferably less than 700 mm Hg, and most preferably less than 600 mm Hg. This range of vacuum pressures is well within the normal operating ranges provided by readily available and inexpensive light duty commercial vacuum pumps that can be incorporated into the systems of the present invention, as well conventional plant-wide vacuum lines that are provided and readily available within most institutional operating facilities.

As shown in FIG. 2C, the next step in operation of system 100 involves re-establishing pneumoperitoneum by again increasing the CO₂ insufflation pressure. This lifts abdominal wall 205, which in the present example serves as the moveable structure. The lifting motion of abdominal wall 205 transmits forces through manifold 110 to liver 215 that thereby also moves. In this manner, liver 215 is simply, safely and atraumatically lifted away from stomach 220, providing ready visualization of, and working access to, gall bladder 230 such that the surgeon may carry out the desired interventional procedure.

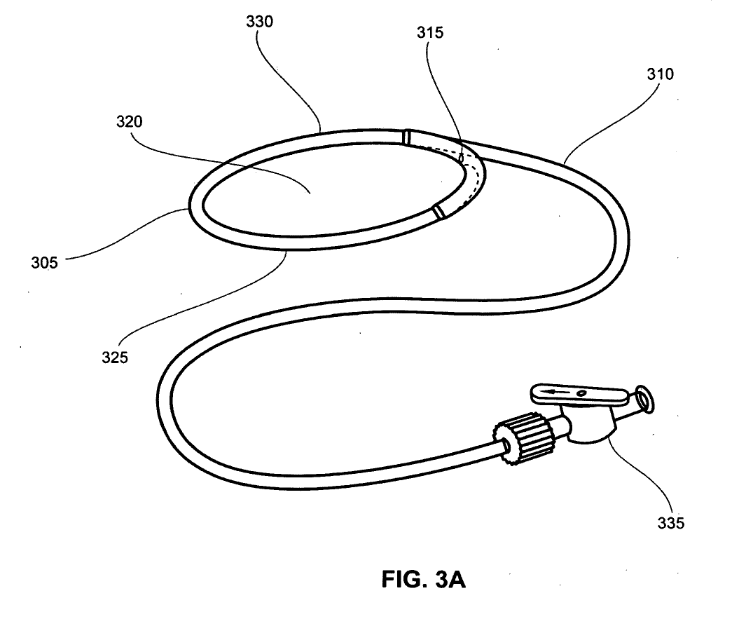

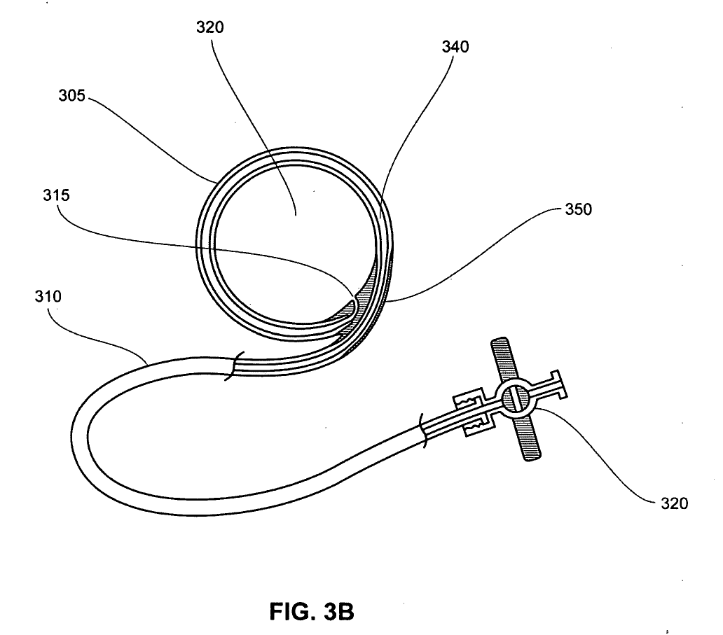

In another embodiment, illustrated in FIG. 3A, manifold 305 consists of a tubular structure that is preferably produced from soft, flexible material. Manifold 305 is configured in the shape of a loop at the distal end of, and more simply as an integral extension of, vacuum tube 310, which is also produced from soft, flexible material. Manifold 305 is configured having a single vacuum passage 315 positioned at the distal, open end of vacuum tube 310, providing a vacuum communication member having a continuous open channel from the remote vacuum source (not shown) to evacuation space 320, consisting of the central, open portion of the loop-shaped structure. In this embodiment, the bottom surface 325 designed to contact the organ to be manipulated, and top surface 330 designed to contact the moveable structure each are formed as a line of contact that is established between the outer surface of the loop-shaped tubular structure when brought into contact with the organ and moveable structure, respectively. Optional valve 335 allows the operator to control vacuum actuation of the device.

A top, cross sectional view of manifold 305 is shown in FIG. 3B. In this view, the continuous vacuum channel 340 passing through vacuum tube 310 and terminating at vacuum passage 315 that communicates with evacuation space 320 is readily apparent. Also shown is optional slip collar 350, a small component to which the distal end of manifold 305 is fixedly attached to create vacuum passage 315, but through which the proximal portion of the loop-shaped structure is slidably and frictionally engaged. Using this optional design feature, the diameter of the loop-shaped structure, and hence the contact surface area of vacuum, can be easily adjusted by the operator, either before inserting the device into the patient or thereafter, by simply sliding the slip collar proximally or distally along vacuum tube 310.

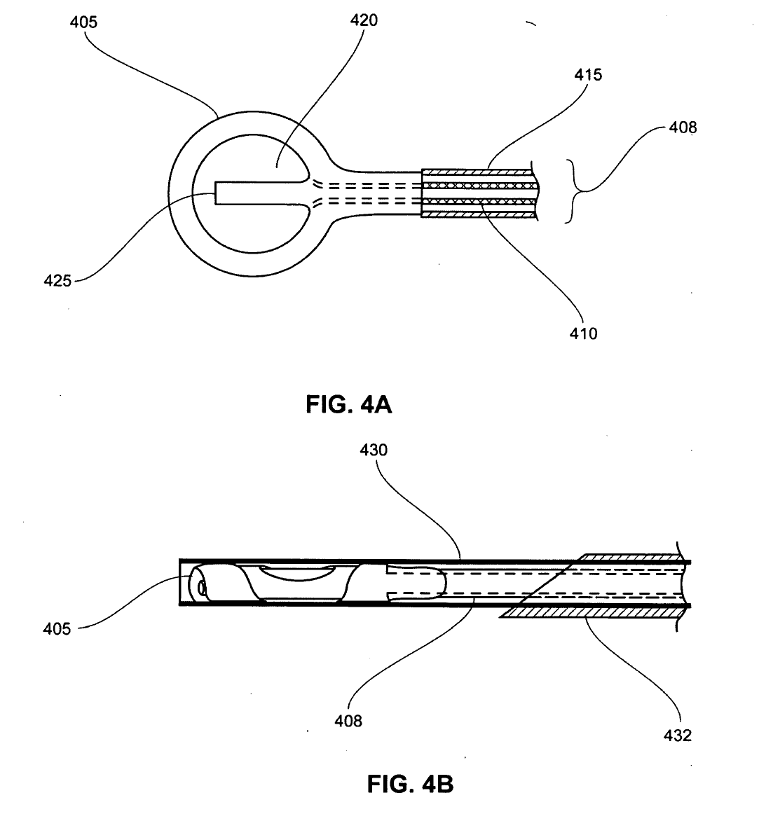

Another embodiment of the present invention is illustrated in FIG. 4. As shown in FIG. 4A, in this case the manifold consists of expandable balloon structure 405 formed in the shape of a ring or doughnut. Expandable balloon 405 is attached to the distal end of a multi-lumen tube 408, as shown. Expandable balloon 405 can be made from any thin sheet or fabric-like biocompatible material that is substantially impermeable such that it can be inflated and deflated by the addition and removal, respectively, of any suitable operating fluid, such as air, inert gas, liquid and the like. Inner vacuum tube 410 is positioned inside and isolated from communicating with outer pressure delivery tube 415. Inner vacuum tube 410 provides a vacuum communication member to vacuum evacuation space 420 via vacuum passage 425, as described previously. In this embodiment, the manifold is configured for insertion into the patient's body in the deflated (pre-deployed) configuration, and it may be rolled, folded or otherwise minimized in size, greatly reducing the device profile prior to deployment. FIG. 4B shows how the expandable balloon, in the deflated (pre-deployed) configuration, may be rolled up around the end of the multi-lumen tube and placed inside flexible delivery catheter 430 that protects the device and allows it to be easily passed through trocar 432 for insertion into the patient's body. Once inside the body, expandable balloon 405 is actuatingly inflated (deployed) by the operator by filling the balloon with the operating fluid using pressure delivery tube 415. After expandable balloon 405 is inflated, it may be used in substantially the same manner as described previously (e.g. FIG. 2) and explained in greater detail below.

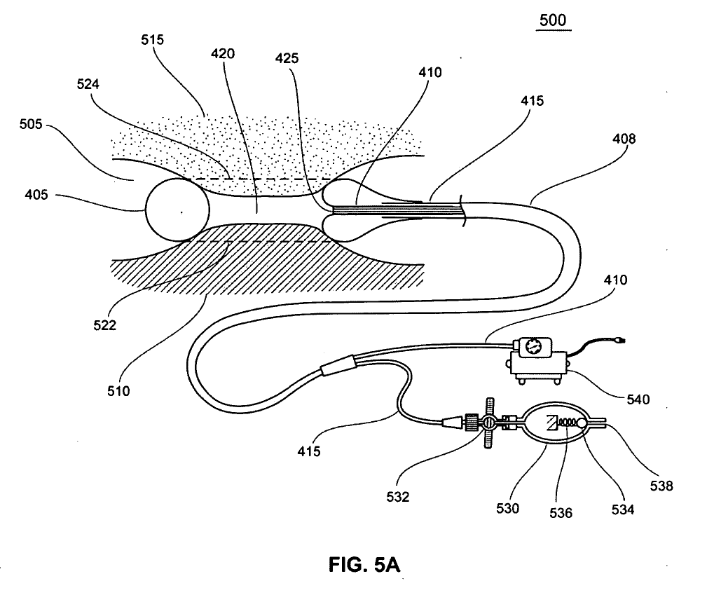

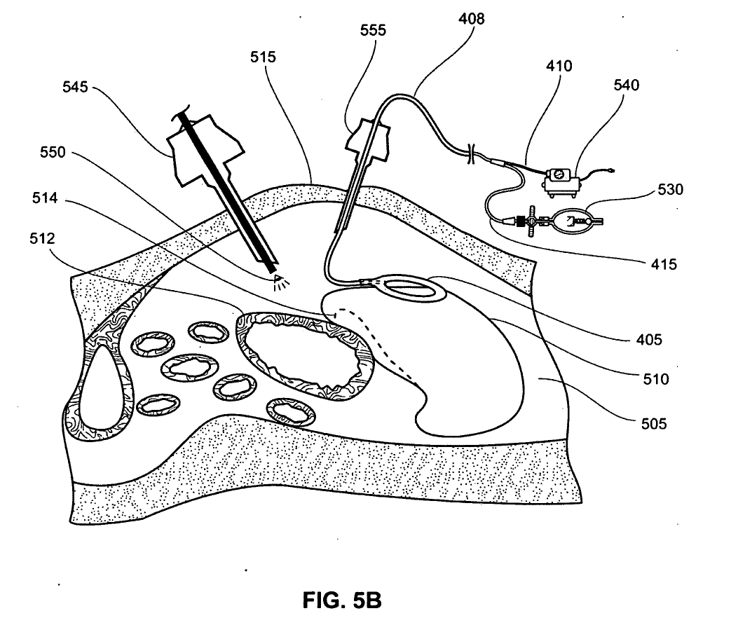

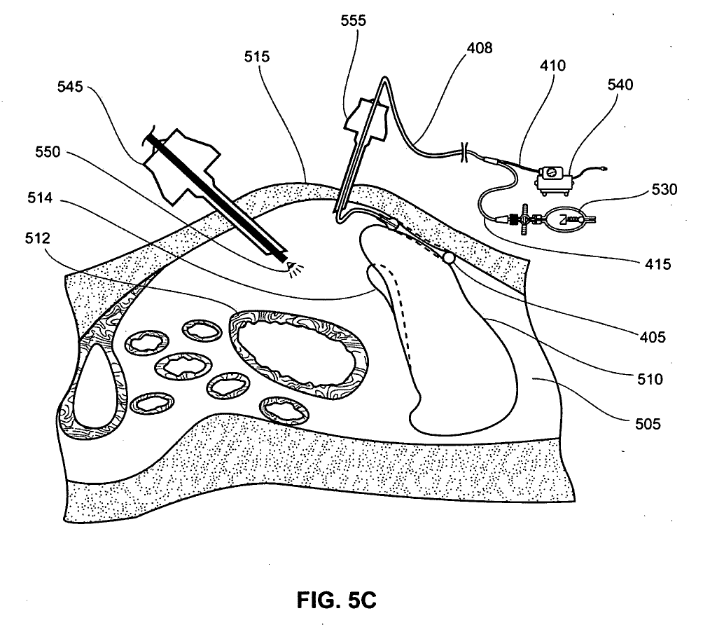

FIG. 5 illustrates detailed operation of a system 500 according to the present invention, in this case for the device embodiment described in FIG. 4. In this example, as described previously, the interventional procedure to be performed is a cholecysectomy and the abdominal wall serves as the moveable structure that will be used to manipulate the target organ. FIG. 5A illustrates close up details of the deployed device configuration within the patient's body. Expandable balloon 405 is positioned inside abdominal cavity 505 between liver 510 and abdominal wall 515. Balloon 405 is positioned at the distal end of multi-lumen tube 408 and has previously been inflated and the pressure has been reduced within evacuation space 420 by actuating the remote vacuum source, causing both liver 510 and abdominal wall 515 to become temporarily attached to balloon 405 along contact surfaces 522 and 524, respectively. To inflate balloon 405 as shown, pressure delivery tube 415 is configured having squeeze ball 530 positioned at its proximal end. Squeeze ball 530 serves as a reservoir, being filled with a suitable working fluid (e.g. air), such that when valve 532 is in the open position and the operator compresses squeeze ball 530, flow ball 534 (which is held by spring 536) seals against opening 538, causing air to be forced distally through pressure delivery tube 415 and into balloon 405. This process may be repeated any number of times wherein the operator may close valve 532 after squeeze ball 530 is compressed, and then by releasing squeeze ball 530, the vacuum within squeeze ball 530 forces flow ball 534 to compress spring 536, allowing air to flow into and refill squeeze ball 530. The operator may then open valve 532 and repeat the process, effectively pumping up balloon 405 to the desired and controlled amount of positive pressure. By leaving valve 532 in the closed configuration after pumping, the balloon then remains in the inflated configuration. It should be appreciated by those skilled in the art that any number of well established methods and devices may be used to inflate balloon 405 in a similar controlled fashion, e.g. compressors, piston pumps, pressure cylinders, and the like, whether manually or automatically controlled, may be used.

Also shown in FIG. 5A is remote vacuum source 540, that when actuated by the operator reduces the pressure within vacuum tube 410, thereby reducing the pressure and generating a controlled vacuum pressure within evacuation space 420 via vacuum passage 425. Although a standalone electrically powered mechanical vacuum pump is depicted here, it should be recognized by those skilled in the art that any number of well established methods for evacuation may be used, e.g. various types of mechanical or other vacuum pumps (whether AC, DC or manually powered) may be used, or alternatively in many medical facilities there may be centralized remote facilities that provide readily available local connections to vacuum lines to which the present system may be connected. In either case, it should also be obvious that various vacuum lines, connections, gauges, switches, sensors, pressure controls, etc., well known to those skilled in the art may be incorporated into systems of the present invention.

FIG. 5B provides an overview of system 500 along with a schematic cross sectional view of a patient, further illustrating certain features as well as methods of usage of the present embodiment. In the case of a typical laparoscopic interventional procedure, the patient's abdominal cavity 505 is usually insufflated by inserting a Veress needle (not shown) through which pressurized gas, typically CO₂, is passed, causing the abdominal wall 515 to lift and thereby creating a working space, as shown. Typically, a trocar is then inserted through abdominal wall, such as first trocar 545, and an endoscopic or laparoscopic camera, such as camera 550, is inserted into the abdominal space to provide the surgeon with direct visualization within the working space, internal organs, etc., such as stomach 512 and gall bladder 514. As illustrated, it is typically not possible for the surgeon to access the gall bladder 514 without some means of lifting, retracting, re-positioning or otherwise manipulating the liver 510. In one method of deploying the device of the present invention, second trocar 555 is inserted through the abdominal wall, and the device, that is initially provided in the pre-deployed configuration within a flexible delivery catheter (as shown in FIG. 4B), is inserted into abdominal cavity 505. Upon retraction of the outer sheath of the flexible delivery catheter (not shown), expandable balloon 405, positioned at the distal end of multi-lumen tube 408, is released from the delivery device and can be deployed as described previously. In most cases, prior to inflation of balloon 405, the surgeon would typically use a grasper or other tool to move and position the balloon on top of liver 510, as shown in FIG. 5B.

Balloon 405 may then be inflated by actuating the remote pressure delivery source, as previously described. At this point, the surgeon would reduce the CO₂ insufflation pressure within abdominal cavity 505, thereby lowering abdominal wall 515 down onto the top surface of balloon 405. Deployment and actuation of the device may then proceed as described previously with respect to FIG. 5A. As shown in FIG. 5C, when abdominal cavity 505 is then re-insufflated by increasing the CO₂ pressure, abdominal wall 515 is again lifted. Balloon 405 and liver 510, which are now temporarily attached to abdominal wall 515, are thereby also lifted. This safely and atraumatically creates a working space where the surgeon has a clear view of gall bladder 514 via camera 550.

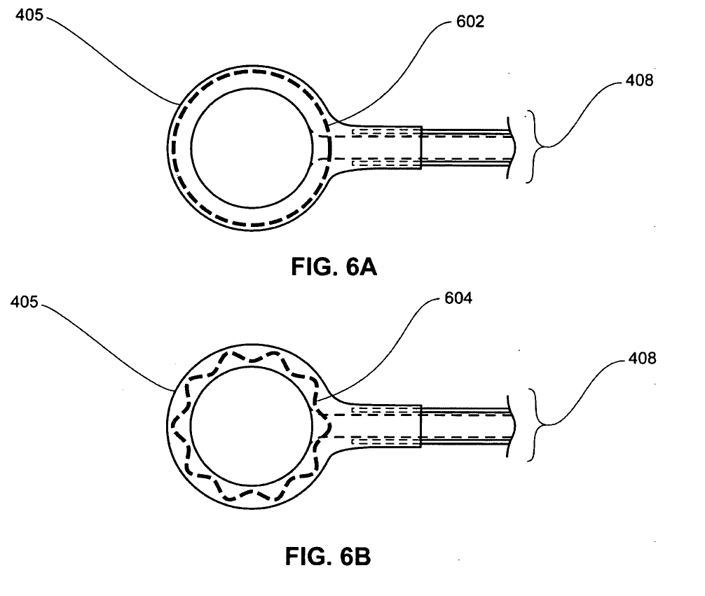

Additional features and mechanisms may be incorporated into devices of the present invention to aid in easy deployment, simplify grasping, positioning and actuation of the device, etc. For example, considering the embodiment described in FIGS. 4 and 5, where the device is initially delivered to the patient's body in a collapsed, pre-deployed configuration, it may be desirable to incorporate one or more highly flexible, self-expanding mechanical elements inside or contained within the wall of balloon 405. As shown in FIG. 6A, for example, balloon 405 may contain internal wire 602 formed in the shape of a loop, that is made from a highly flexible material such as superelastic NiTi alloy or the like. Wire 602 is capable of being elastically deformed into a collapsed condition when balloon 405 is rolled up and loaded into the delivery catheter (e.g. FIG. 4B), but returns to its original loop shape in a self-actuating manner when released from the delivery catheter, thereby automatically unfolding balloon 405 and serving as a support frame that allows balloon 405 to retain its shape and be more easily manipulated prior to inflation. Similarly, as shown in FIG. 6B, the self-expanding internal frame may have other shapes, such as wavy wire 604, that further help to initially expand and/or support balloon 405 prior to and during deployment.

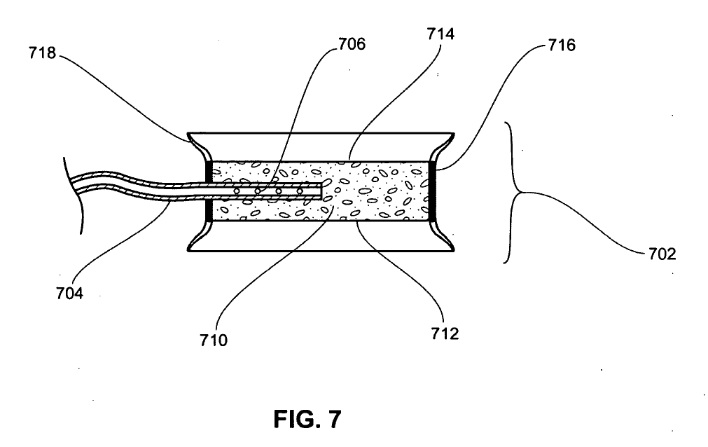

Another embodiment of the present invention is illustrated in FIG. 7. This device is similar to the device illustrated in FIG. 4 and operates substantially similarly that the operation illustrated in FIG. 5. In this embodiment, assembly 702 is positioned at the distal end of vacuum tube 704 and is designed to be inserted into the patient's body and used to manipulate organs as previously described. Assembly 702 consists of a central portion 710, having bottom surface 712 configured for contacting tissues or organs to be manipulated, top surface 714 configured for contacting a moveable structure, and perimeter 716. Central portion 710 is made from a flexible, open cell biocompatible foam or other similar material that is flexible, highly porous and compressible, and formed in the shape of a thin sheet, disk, pad, plate or the like. A wide variety of suitable materials are commercially available, such as polymer foams, foamed silicones, rubbers, sponges, and the like. Owing to it's flexible, open porous nature, central portion 710 is capable of being compressed to remove the air filling its open passages, and in this manner being provided in a collapsed, pre-deployed configuration for insertion into the patient's body, in a manner similar to FIG. 4B. However, when released from its compressed, collapsed state, the material expands in a self-actuating manner as air (or the insufflation gas) flows back into and re-inflates the porous structure. This behavior is similar to that employed in lightweight and highly compressible self-inflating mattress products sold in the backpacking and camping gear markets. The primary advantage of this embodiment over that illustrated in FIG. 4 is that the structure is self-inflating, i.e. no external pressure source or pressure delivery mechanism is needed to inflate the device during initial deployment.

Perimeter 716 is preferably made from a thin, vacuum impermeable coating, fabric or the like, that completely surrounds and covers all external surfaces of central portion 710 that are not intended to either contact tissue or the moveable structure during device operation. Alternatively, perimeter 716 may be formed by selectively heat sealing or partially melting the outer surface of the porous material comprising central portion 710. During use, after insertion into the patient's body and self-actuating inflated expansion of central portion 710, as described above, the external vacuum source (not shown) is actuated. Reduced pressure is thereby delivered to central portion 710 via vacuum tube 704 and vacuum passages 706. Because perimeter 716 is vacuum impermeable, the internal vacuum generated inside central portion 710 produces a suction effect at each of tissue contacting surface 712 and moveable structure contacting surface 714. This suction creates forces that act to draw toward and temporarily attach assembly 702 to each of the tissue/organ to be manipulated and the moveable structure, respectively. Optional seals 718, which may be produced from soft, flexible impermeable material such as rubber, fabric, or the like, may be provided and configured at the top and bottom surfaces of assembly 702 in order to assist with the initial contact and suction effects needed to achieve temporary attachment to each contacting surface during device actuation. Some advantages of this embodiment are that the device is compressible to a very small profile in the pre-deployed configuration for insertion into the patient's body, the structure is self-inflating on initial deployment, and the surface areas provided for contacting each of the tissue/organ to be manipulated and the moveable structure may be designed to be significantly larger than in the case of an inflatable balloon. This further reduces stress concentrations on tissues during vacuum actuation, thereby providing a safer, more atraumatic and easily releasable temporary attachment that allows for improved organ manipulation.

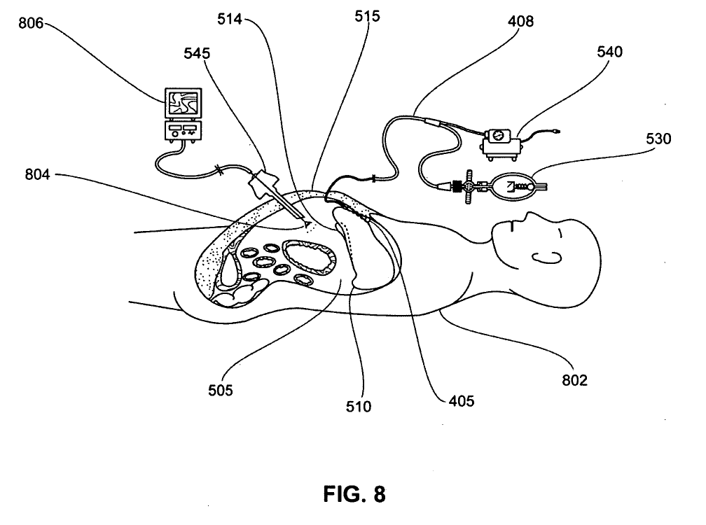

FIG. 8 illustrates one method of using a system of the present invention within a patient 802, along with complimentary videoscopic camera 804 and monitor 806. This example illustrates the situation likely employed when performing a laparoscopic interventional (e.g. a diagnostic or therapeutic) procedure where the liver 510 needs to lifted or manipulated. In this case, the device such as that described and illustrated with reference to FIG. 4 has been deployed and actuated as described and illustrated with reference to FIG. 5. After deployment and actuation, as shown, the patient's abdominal cavity 505 is insufflated and the patient's abdominal wall 515 and liver 510 have been safely and atraumatically lifted by inflatable balloon 405, thereby creating an effective working space and good visualization of the operative field for the surgeon to perform the desired intervention, e.g. a laparoscopic cholecysectomy wherein gall bladder 514 is removed. In this particular example, it is illustrated that second trocar 555 was needed only briefly to insert the device into the patient and has been optionally removed after insertion of the device. This advantageously leaves only multi-lumen tube 408 passing through the incision in abdominal wall 515. This may be accomplished, for example, by temporarily disconnecting tube 408 from pressure source 530 and vacuum source 540, removing second trocar 555 from the body and sliding off the end of tube 408, and then re-connecting tube 408 to pressure source 530 and vacuum source 540. It may be desirable in some cases to employ a small optional seal around tube 408 where it passes through abdominal wall 515 in order to prevent leakage of the insufflation pressure. At the end of the procedure, tube 408 may again be disconnected from pressure source 530 and vacuum source 540 and the device may be removed from the patient's body through first trocar 545. In this manner, the incision made for inserting second trocar 555 in order to insert and deploy the device is less invasive to patient.

There are a variety of minor modifications to the devices and operational methods that can be employed to allow the present invention to be used without requiring any separate incisions or trocars. For example, in one such alternative (not shown), it is possible to use the device of the present invention with a trocar that will used for other purposes, such as trocar 545. In this case, the device is first inserted through abdominal wall 515 and into the patient's abdominal cavity through an incision made in abdominal wall 515, but prior to placing trocar 545 into the incision. Trocar 545 may then be placed through the abdominal wall with tube 408 routed, e.g. adjacent the outside surface of the trocar, therefore not requiring use of the working channel of the trocar. Because tube 408 is very small and flexible, it is able to conform to the interface between trocar 545 and abdominal wall 515 and therefore the rate of leakage of insufflation pressure using this configuration, if any, is relatively low.

In yet another alternative embodiment (not shown), it is also possible to incorporate one or more small, inline shutoff valves (ideally there are separate shutoff valves for each of the pressure and vacuum lines), along the length of tube 408 at or near the location where tube 408 attaches to balloon 405. Tube 408 may then be detachably connected to said shutoff valve(s). In this manner, after insertion of the device into the patient's body and subsequent pressurized deployment, followed by vacuum actuation to temporarily attach balloon 405 to both liver 510 and abdominal wall 515, said shutoff valve(s) can be closed. This will maintain balloon 405 in the deployed (inflated) and vacuum actuated configuration, even after tube 408 is detached from the shutoff valve(s) and removed from the body. In this manner, it is possible to lift and retract the liver or other organs without requiring a continuously active connection between the device and pressure source 530 and vacuum source 540. If desired or necessary to reposition or adjust the device, it is possible at any time to re-insert tube 508 into the patient via any previously placed trocar and re-connect to the shutoff valve(s).

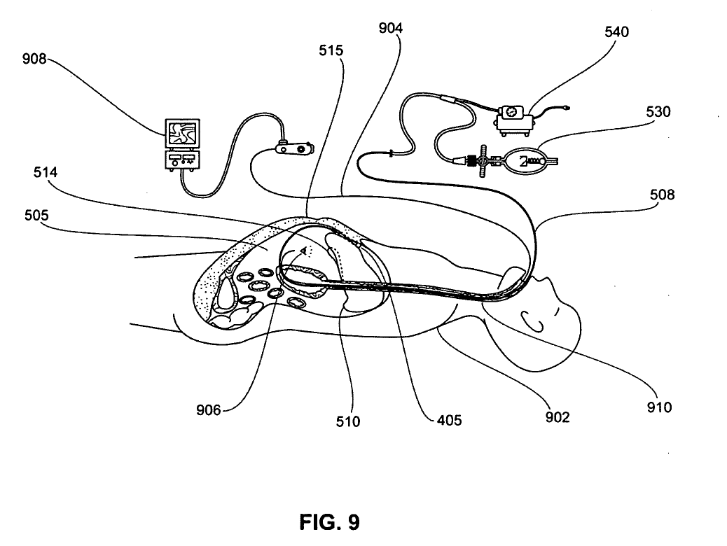

An alternative method of using the systems of the present invention within a patient 902 is illustrated in FIG. 9. This example illustrates the situation likely employed when performing a transgastric endoscopic interventional (i.e. NOTES) procedure where the liver needs to lifted or manipulated, and there is a need for even smaller and less invasive tools. In this case, a flexible endoscope 904 is first inserted into the patient's esophagus 910 and advanced into the patient's stomach, through the gastrointestinal wall, and into abdominal cavity 505. After insufflation of the patient's abdominal cavity, and under visualization provided by the endoscopic video camera 906 on monitor 908, a device such as that described and illustrated with respect to FIG. 4 is deployed into the abdominal cavity. The steps of deploying the inflatable balloon 405 onto the surface of the liver 510, de-insufflating the abdominal cavity 505 to lower the abdominal wall into the top surface of the balloon, vacuum actuating the device to temporarily attach the liver and abdominal wall to the bottom and top surfaces of the balloon, respectively, and subsequently re-insufflating the abdominal cavity are substantially similar the steps described previously with reference to FIG. 5. After deployment and actuation, as shown, the patient's abdominal cavity 505 is insufflated and the patient's liver 510 has been safely and atraumatically lifted, thereby creating an effective working space and good visualization of the operative field for the surgeon to perform the desired intervention, e.g. in this case a translumenal endoscopic cholecysectomy wherein gall bladder 514 is removed through the patient's mouth without any external incisions.

In practice, it is possible but not considered necessary for the device of the present invention to be deployed through the working channel of flexible endoscope 904. As shown in FIG. 9, it is possible to route multi-lumen tube 508 on the outside and along the exterior wall of flexible endoscope 904, in order to preserve the working channel for other instruments needed by the surgeon to perform the desired diagnostic or therapeutic interventional procedure. The fact that only a small, flexible multi-lumen tube is needed to deploy and actuate the devices of the present invention is considered a significant advantage over other methods of lifting and manipulating organs based on mechanical leverage. It should be obvious to those skilled in the art that substantially the same methods and devices illustrated in FIG. 9 may be employed in other types of NOTES procedures, such as those utilizing transanal or transvaginal access to the abdominal cavity.

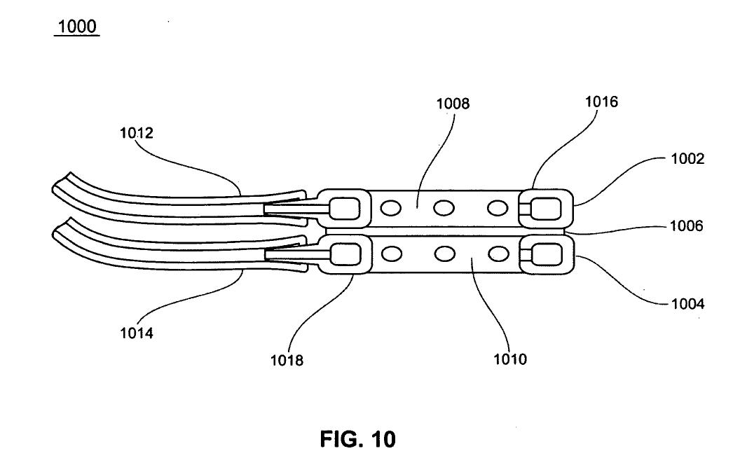

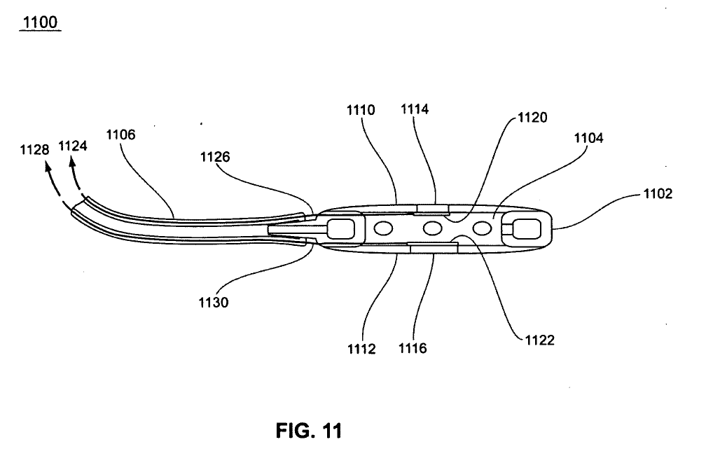

Various additional features and mechanisms may be optionally incorporated into the devices and systems of the present invention to provide greater design flexibility, enhanced functionality, ease-of-use, improved safety, etc. For example, another embodiment of the present invention is illustrated in FIG. 10. In this embodiment, it is desirable to provide mechanisms for independently and selectively controlling the actuation of the manifold with regard to communicating vacuum to each of the contacting surfaces. Such actuation (and the resulting independently controllable surface attachments) can thereby be performed either simultaneously (as described above) or sequentially, as may desired to most effectively utilize the device for certain specific intended purposes. This option may be readily incorporated, for example, by providing more than one evacuation space configured within the manifold, each having its own controllable vacuum communication member connected with the vacuum source, as well as its own vacuum port(s) for communicating vacuum between the respective evacuation space and contacting surface. As shown in FIG. 10, manifold assembly 1000 consists of first manifold portion 1002 and second manifold portion 1004, each of which is substantially similar to the manifold described in FIG. 1B, except that separator 1006 provides an impermeable barrier that prevents vacuum communication between first evacuation space 1008 and second evacuation space 1010. First manifold portion 1002 and second manifold portion 1004 are configured having first vacuum communication member 1012 and second vacuum communication member 1014 connected thereto, respectively, each of which is operatively connected to, and independently actuatable by, the vacuum source (not shown). The circular opening within top surface 1016 of first manifold portion 1002 provides a first vacuum port, and the circular opening within bottom surface 1018 of second manifold portion 1004 provides a second vacuum port, each of which is designed and configured for communicating vacuum from the respective evacuation space to the tissue/organ and/or moveable structure to which attachment is desired.

Alternatively, as illustrated by another embodiment shown in FIG. 11, it is also possible to provide for independent and selective actuated attachment at each contacting surface by having a single, common evacuation space while incorporating one or more valves, switches, sensors or other controllable mechanisms for operably opening and/or closing the vacuum ports for communicating vacuum between the evacuation space and at least one of the contacting surfaces. As shown in FIG. 11, manifold assembly 1100 consists of ring-shaped manifold 1102 that is substantially similar to the manifold described in FIG. 1B, having evacuation space 1104 therewithin, and vacuum communication member 1106 operably connected thereto, and being actuatably connected to the remote vacuum source (not shown). In this example, manifold 1102 is further configured having top plate 1110 and bottom plate 1112 covering a portion of the top and bottom surfaces of manifold 1102, respectively, and thereby serving as the contacting surfaces. Top plate 1110 is configured having first vacuum port 1114 passing through it, and bottom plate 1112 is configured having second vacuum port 1116 passing through it, where both vacuum ports communicate vacuum between evacuation space 1104 and the tissue/organ and/or moveable structure to which the respective surface will be attached. Note in this example that first vacuum port 1114 and second vacuum port 1116 optionally have different diameters, providing different contact surface areas of vacuum communication, and hence allowing different holding forces to be generated at each contacting surface. Note also in this example, that first vacuum port 1114 is configured having first slidable valve 1120, and second vacuum port 1116 is configured having second slidable valve 1122, where each of said valves 1120 and 1122 is designed and configured to be capable of independently controllable opening and closing of the respective vacuum port when actuated by the user. In this example, actuated opening of first valve 1120 is effected by pulling proximally 1124 on first control wire 1126, which is operably connected to valve 1120 and remotely controlled by the operator. Similarly, actuated opening of second valve 1122 is effected by pulling proximally 1128 on second control wire 1130, which is operably connected to valve 1122 and remotely controlled by the operator. Valves 1120 and 1122 may optionally be spring loaded and biased in the closed configuration such that remote release of the respective control wire by the operator causes the valve to automatically close.

Note that in the embodiment illustrated in FIG. 11, there is shown only a single vacuum port configured on each contacting surface, namely first vacuum port 1114 and second vacuum port 1116. However, it should be recognized that any number of multiple vacuum ports having either uniform sizes or varied sizes may be configured and distributed substantially across the respective contacting surfaces in order to optimize the magnitude and spatial distribution of the holding forces. Accordingly, various other the operable mechanisms and configurations may be optionally incorporated for selectively controlling the opening and/or closing of the one or more vacuum ports in communication with common evacuation space 1104. For example, when there are multiple vacuum ports positioned on a contacting surface, it may be possible to partially or selectively open/close only a desired fraction of the total contact surface area of vacuum that is available, thereby providing additional independent control over the magnitude and spatial distribution of the holding forces generated on each contacting surface.

To illustrate the useful benefit of incorporating optional independent actuation, for example, in some situations it may desirable to initially attach the manifold to a first tissue to form a subassembly at a first position, then move the subassembly (i.e. the manifold with the first tissue attached thereto) to another second position at which point it may then be attached to another tissue to produce a completed, joined assembly (i.e. the manifold temporarily and releasably attached at two or more contacting surfaces to tissues, organs, moveable structures, etc.) within the body. Similarly, the second tissue to be attached to the initially formed subassembly at the first position may itself be independently moved by the clinician toward the previously formed subassembly, and then attached thereto to form a completed, joined assembly located at the first position. In either case, the subsequent lifting, positioning, retracting or otherwise manipulating of either attached tissue may be accomplished by manipulating either the other attached tissue or the manifold itself.

### EXAMPLE

The present invention has been successfully reduced to practice via a number of different embodiments, as described above. In one example, described here, a manifold according the embodiment shown in FIG. 1B was produced by cutting and machining a polymer tube into a ring shape having an outer diameter of 4.8 cm, an inner diameter of 4.1 cm and a height of 0.5 cm. A flexible silicone tube was used as a vacuum communication member, having an outer diameter of 0.38 cm. The tube was connected at its distal end to the manifold using a threaded nipple connector that passed completely through the circumferential wall of the manifold, providing a vacuum passage through the wall of the manifold into the interior space within the ring, which thereby served as the evacuation space. The proximal end of the flexible silicone tube was connected to a shutoff valve that was then connected to the vacuum side of an AC powered vacuum pump capable of achieving an ultimate vacuum pressure of less than 300 mm Hg (absolute).

To demonstrate the operational methods and functional capabilities of the present invention, the organ to be manipulated and the moveable structure of the present invention were both simulated using water filled balloons. In this experiment, each balloon was filled with approximately 1 liter of water and then sealed, weighing approximately 1 kg.

The manifold was first placed atop one of the balloons (the bottom balloon, representing the patient's liver), thereby simulating deployment of the device within a patient's insufflated abdominal cavity and positioning of the device on top of the liver, as illustrated in FIG. 2A. The second balloon (the top balloon, representing the patient's abdominal wall), being held and supported from above, was carefully lowered onto the top surface of the manifold, thereby simulating lowering of the abdominal wall by reducing the insufflation pressure within the abdominal cavity, as illustrated in FIG. 2B. At this point, the vacuum pump was energized and the shutoff valve was opened, thereby gradually reducing the pressure inside the evacuation space created within the manifold ring and between the surfaces of the top and bottom balloons. Evacuation caused both balloons to be drawn in and become temporarily attached to the top and bottom surfaces of the manifold. The vacuum pressure was set at approximately 200 mm Hg and the shutoff valve was then closed, thereby maintaining the vacuum pressure within the evacuation space and keeping both balloons joined to the manifold.

To simulate lifting of the abdominal wall by re-insufflation of the abdominal cavity, as illustrated in FIG. 2C, the topmost balloon was lifted from above. In so doing, the bottom balloon, which was connected via the vacuum actuated manifold, was also lifted completely off the workbench, thereby simulating the lifting and retraction of the patient's liver. The lifted configuration remained stable for as long as the vacuum pressure was maintained. After lowering of the balloons and upon release of the vacuum by opening the shutoff valve, the balloons were easily disengaged from the manifold with no visual evidence of damage.

According to the methods of the present invention, this example clearly illustrates successful operation of one embodiment of the devices and systems of the present invention, demonstrating the ability to deploy, control, actuate and successfully utilize a vacuum actuated manifold of the present invention in the intended manner. Furthermore, given this experiment was carried out within a performance range designed to be useful for a wide variety of interventional procedures, this example further demonstrates the present invention is capable of providing sufficient holding force and lifting capacity to manipulate heavy organs and tissues within the body, such as the liver, and wherein the maximum pressure exerted on the target organ/tissue is limited by design to prevent trauma or unintended damage.

I claim:

1. A system for manipulating tissue comprising: a manifold configured for insertion into the body of a patient, an external vacuum source, and a vacuum communication member operably connecting said manifold to said vacuum source, wherein said manifold further comprises at least a first surface configured for contacting tissue to be manipulated, at least a second surface configured for contacting at least one other structure, at least one evacuation space in operable communication with said first surface via at least a first vacuum port and said second surface via at least a second vacuum port.

2. A device for manipulating tissue inside a patient comprising a manifold wherein said manifold further comprises at least a first surface configured for contacting tissue to be manipulated, at least a second surface configured for contacting at least one other structure, and at least one evacuation space in operable communication with said first surface via at least a first vacuum port and said second surface via at least a second vacuum port.

3. A device of claim 2 wherein the manifold is selected from the group consisting of rigid, flexible and combinations thereof.

4. A device of claim 2 wherein the manifold is initially provided in a collapsed configuration for delivery into the body and is capable of being deployed into an expanded configuration after insertion into the body.

5. A device of claim 2 wherein the manifold comprises an inflatable, substantially ring-shaped balloon.

6. A device of claim 2 wherein the manifold comprises a permeable porous material surrounded by a substantially impermeable perimeter.

7. A method for manipulating tissue inside a patient comprising:
a. providing a manifold into a patient's body, said manifold having at least a first surface configured for contacting tissue to be manipulated, at least a second surface configured for contacting at least one other structure, and at least one evacuation space in operable communication with said first surface via at least a first vacuum port and said second surface via at least a second vacuum port;
b. positioning the manifold such that the first surface is in substantially intimate contact with the tissue to be manipulated and the second surface is in substantially intimate contact with the at least one other structure;
c. operatively reducing the pressure inside the evacuation space to a level sufficient to temporarily adhere both the tissue to be manipulated and the at least one other structure to the manifold; and
d. manipulating the at least one other structure.

8. A method of claim 7 wherein the at least one other structure is selected from the group consisting of a separately provided mechanical component, another body tissue that may be moved by the operator, and combinations of the foregoing.
